(19) United States
(12) Patent Application Publication
(10) Pub. No.: US 2012/0041410 A1
(43) Pub. Date: Feb. 16, 2012

(54) Disposable Absorbent Article Having Soft-Clothlike Backsheet

(76) Inventors: Kimio Ueda, Kobe (JP); Jie TAO, Ashiya-shi (JP)

(21) Appl. No.: 13/283,026

(22) Filed: Oct. 27, 2011

Related U.S. Application Data
(63) Continuation of application No. 09/297,774, filed on May 7, 1999.

(51) Int. Cl. A61F 13/49

(52) U.S. Cl. 604/385.25

(57) ABSTRACT

A disposable absorbent article comprises a containment assembly comprising a topsheet, a backsheet and an absorbent core disposed between the topsheet and the backsheet. The backsheet comprises a nonwoven web positioned at the outermost portion of the absorbent article. The backsheet covers at least a portion of the outermost portion of the absorbent core of the article. The backsheet includes a nonwoven web, which is a spunbonded nonwoven web. The spunbonded nonwoven web has a tensile strength of at least 180 gf/cm in the traverse direction of the disposable absorbent article. The backsheet further comprises a plastic film having an outer-facing surface and a body-facing surface, and the nonwoven web is joined with the outer-facing surface of the plastic film to form a laminate.

### CROSS REFERENCE TO RELATED APPLICATION

This applications is a continuation of application Ser. No. 09/297,774, filed May 7, 1999, which is a National Phase entry and claims priority to PCT/US96/18339, filed Nov. 8, 1996, the substance of which is incorporated herein by reference.

### FIELD OF THE INVENTION

The present invention relates generally to disposable absorbent articles such as disposable diapers and, more particularly, to disposable absorbent articles having soft-clothlike backsheets.

### BACKGROUND OF THE INVENTION

Infants and other incontinent individuals wear disposable absorbent articles such as diapers to receive and contain urine and other body exudates. Absorbent articles function both to contain the discharged materials and to isolate these materials from the body of the wearer and from the wearer's garments and bed clothing. Disposable absorbent articles having many different basic designs are known to the art. It is also known that the exterior of disposable diapers can be covered with a flexible, liquid and vapor impervious sheet, which prevents any absorbed liquid from passing through the diaper and soiling adjacent articles such as clothing, bedding and the like. These outer covers, generally referred to as backsheets, are often constructed from fluid impervious films such as polyethylene. Although such backsheets do prevent liquid from passing through the diaper, they also can make the diaper feel hot and uncomfortable to wear because of their impermeability to air and/or moisture.

Backsheets which are pervious to vapor are generally known as breathable backsheets and have been described in the art. In general, these backsheets are intended to allow the passage of vapor through them while retarding the passage of liquid. The conventional breathable backsheets are usually made of microporous thin plastic films. For example, U.S. Pat. No. 3,156,242 issued to Crowe, Jr. on Nov. 10, 1964 teaches the use of a microporous film as a breathable backsheet. U.S. Pat. No. 3,881,489, issued to Hartwell on May 6, 1975, teaches a breathable backsheet comprising in combination two layers. The first layer is a low void volume perforated thermoplastic film and the second layer is a porous high void volume hydrophobic tissue.

Some recent disposable diapers use cloth-like backsheets to provide a visual breathability and an improved natural appearance and/or impression. A typical structure of such cloth-like backsheets comprises a nonwoven web joined to the outer-facing surface of a microporous thin plastic in order to form a laminate.

However, such cloth-like backsheets are not completely satisfactory to consumers because they are either stiff, bulky or have a rough surface. For example, the friction between the backsheet and the wearer's skin often causes skin rash on the wearer's legs. Further, the friction between the backsheet and the wearer's clothes generates a noise and is also uncomfortable to the wearer. It is believed that such frictions are caused by excessive crispness and roughness at the surface of backsheets.

### SUMMARY OF THE INVENTION

Briefly stated, the present invention relates to a disposable absorbent article. In one aspect of the invention, the disposable absorbent article comprises a containment assembly comprising a topsheet, a backsheet and an absorbent core disposed between the topsheet and the backsheet. The backsheet comprises a nonwoven web positioned at the outermost portion of the absorbent article, for covering at least a portion of the outermost portion of the absorbent core of the article. The backsheet has a hand value of Koshi of less than about 11.0, a hand value of Shari of from about 5.0 to about 7.0, and a hand value of Fukurami of less than about 0.5. Preferably, the backsheet has a fuzz level of less than about 0.24 mg/cm². More preferably, the nonwoven web is a spunbonded nonwoven web. In a preferred embodiment, the spunbonded nonwoven web has a tensile strength of at least 180 gf/cm in the traverse direction of the disposable absorbent article.

In another aspect of the invention, the disposable absorbent article comprises a containment assembly comprising a topsheet, a backsheet and an absorbent core disposed between the topsheet and the backsheet. The backsheet comprises a nonwoven web positioned at the outermost portion of the absorbent article, for covering at least a portion of the outermost portion of the absorbent core of the article. The nonwoven web is a spunbonded nonwoven web comprising spunbonded bi-component plastic fibers. Preferably, the spunbonded nonwoven web is placed in the disposable absorbent article so that the fiber direction of the spunbonded bi-component plastic fibers is aligned with the longitudinal direction of the disposable absorbent article. More preferably, the spunbonded nonwoven web has a tensile strength of at least 80 gf/cm in the traverse direction of the disposable absorbent article. In a preferred embodiment, the nonwoven web has a hand value of Koshi of less than about 16.0, a hand value of Shari of from about 0.5 to about 9.5, and a hand value of Fukurami of less than about 5.0. In a further preferred embodiment, the nonwoven web has a fuzz level of less than about 1.0 mg/cm².

Preferably, the backsheet further comprises a plastic film having an outer-facing surface and a body-facing surface, and the nonwoven web is joined with the outer-facing surface of the plastic film to form a laminate.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings in which like designations are used to designate substantially identical elements, and in which:

FIG. 1 is a plane view of a disposable diaper embodiment according to the present invention having portions cut away to reveal underlying structure, the inner surface of the diaper is facing the viewer;

FIG. 2 is an enlarged, cross-sectional view of one embodiment of a disposable absorbent article comprising a backsheet;

FIG. 3 is a plane view of the sample used for the tensile property measurement;

FIG. 4 is a graph showing the tensile property of the sample;

FIGS. 5A and 5B are schematic diagrams explaining the measurement for the bending property;

FIG. 6 is a graph showing the bending property of the sample;

FIGS. 7A and 7B are schematic diagrams explaining the measurement for the surface roughness;

FIGS. 8A and 8B are schematic diagrams explaining the measurement for the surface friction;

FIG. 9 shows the conditions of the steel plate used for the surface roughness and friction measurements;

FIG. 10 shows the changes of the friction coefficient along the surface of the sample;

FIG. 11 shows the changes of the thickness along the surface of the sample;

FIG. 12 is a plane view of the sample used for the shearing property measurement;

FIG. 13 is a graph showing the shearing property of the sample;

FIG. 14 is a plane view of the sample used for the compression property measurement;

FIG. 15 is a graph showing the compression property of the sample;

FIG. 16 is a schematic diagram explaining the fuzz level measurement.

### DETAILED DESCRIPTION OF THE INVENTION

### 1. Absorbent Article

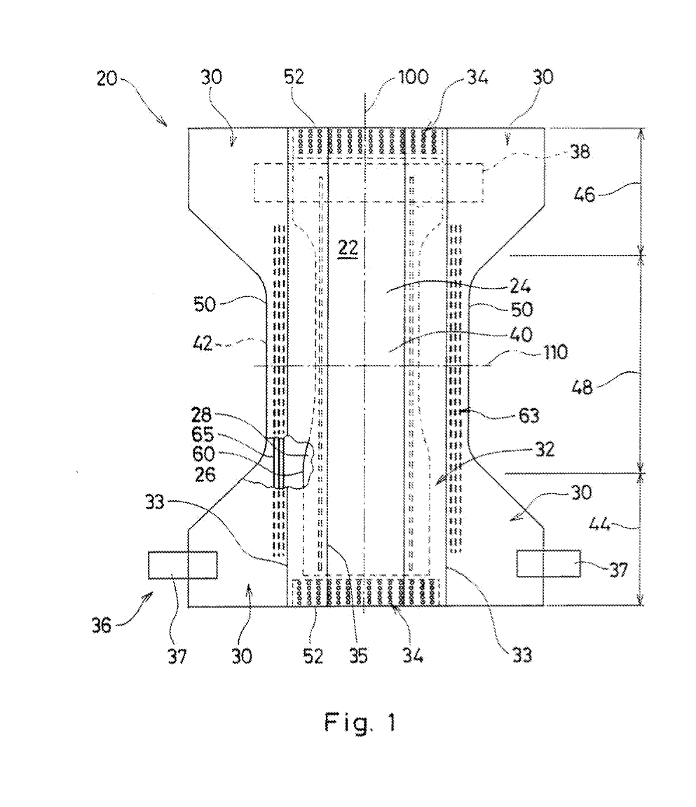

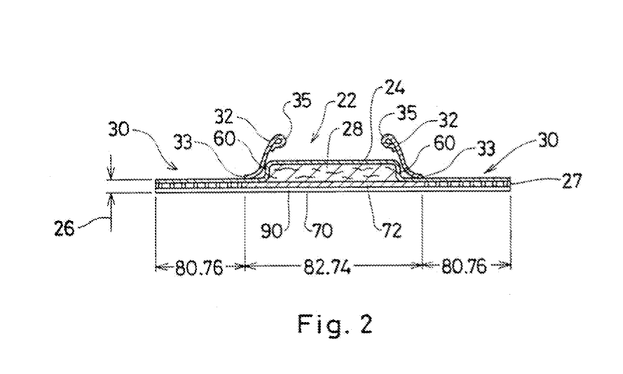

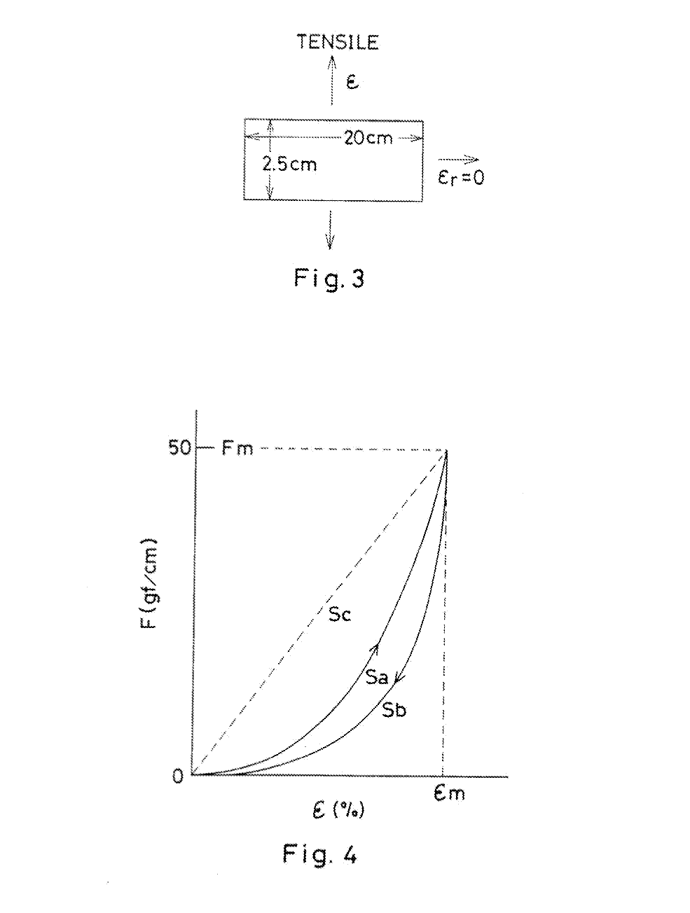

As used herein, the term “absorbent article” refers to devices which absorb and contain body exudates, and, more specifically, refers to devices which are placed against or in proximity to the body of the wearer to absorb and contain the various exudates discharged from the body. The term “disposable” is used herein to describe absorbent articles which are not intended to be laundered or otherwise restored or reused as an absorbent article (i.e., they are intended to be discarded after a single use and, preferably, to be recycled, composted or otherwise disposed of in an environmentally compatible manner). A “unitary” absorbent article refers to absorbent articles which are formed of separate parts united together to form a coordinated entity so that they do not require separate manipulative parts like a separate holder and liner. A preferred embodiment of an absorbent article of the present invention is the unitary disposable absorbent article, diaper 20, shown in FIG. 1. As used herein, the term “diaper” refers to an absorbent article generally worn by infants and incontinent persons that is worn about the lower torso of the wearer. It should be understood, however, that the present invention is also applicable to other absorbent articles such as incontinence briefs, incontinence undergarments, diaper holders and liners, feminine hygiene garments, training pants, and the like.

FIG. 1 is a plane view of the diaper 20 in its flat-out, uncontracted state (i.e., with elastic induced contraction pulled out) with portions of the structure being cut-away to more clearly show the construction of the diaper 20 and with the portion of the diaper 20 which faces the wearer, the inner surface 40, facing the viewer. As shown in FIG. 1, the diaper 20 preferably comprises a containment assembly 22 comprising a liquid pervious topsheet 24; a liquid impervious backsheet 26 joined to the topsheet; and an absorbent core 28 positioned between the topsheet 24 and the backsheet 26. The absorbent core 28 has a pair of opposing longitudinal edges 60, an inner surface and an outer surface. The diaper preferably further comprises side panels 30; elasticized leg cuffs 32 (each having an inboard edge 35 and an outboard edge 32); elasticized waistbands 34; and a fastening system 36 preferably comprising a pair of securement members 37 and a landing member 38. The backsheet 26 prevents the exudates absorbed and contained in the absorbent core 28 from wetting articles which contact the diaper 20 such as bed sheets and undergarments.

The diaper 20 has an inner surface 40 (facing the viewer in FIG. 1), an outer surface 42 opposed to the inner surface 40, a rear waist region 44, a front waist region 46 opposed to the rear waist region 44, a crotch region 48 positioned between the rear waist region 44 and the front waist region 46, and a periphery which is defined by the outer perimeter or edges of the diaper 20 in which the side edges are designated 50 and the end edges are designated 52. The inner surface 40 of the diaper 20 comprises that portion of the diaper 20 which is positioned adjacent to the wearer's body during use (i.e., the inner surface 40 generally is formed by at least a portion of the topsheet 24 and other components joined to the topsheet 24). The outer surface 42 comprises that portion of the diaper 20 which is positioned away from the wearer's body (i.e., the outer surface 42 is generally formed by at least a portion of the backsheet 26 and other components joined to the backsheet 26). As used herein, the term “joined” encompasses configurations whereby an element is directly secured to the other element by affixing the element directly to the other element, and configurations whereby the element is indirectly secured to the other element by affixing the element to intermediate member(s) which in turn are affixed to the other element. The rear waist region 44 and the front waist region 46 extend from the end edges 52 of the periphery to the crotch region 48.

The diaper 20 also has two centerlines, a longitudinal centerline 100 and a transverse centerline 110. The term “longitudinal”, as used herein, refers to a line, axis, or direction in the plane of the diaper 20 that is generally aligned with (e.g. approximately parallel with) a vertical plane which bisects a standing wearer into left and right halves when the diaper 20 is worn. The term “transverse”, as used herein, are interchangeable and refer to a line, axis or direction which lies within the plane of the diaper that is generally perpendicular to the longitudinal direction (which divides the wearer into front and back body halves).

The topsheet 24 and the backsheet 26 have length and width dimensions generally larger than those of the absorbent core 28. The topsheet 24 and the backsheet 26 extend beyond the edges of the absorbent core 28 to thereby form the periphery of the diaper 20. While the topsheet 24, the backsheet 26, and the absorbent core 28 may be assembled in a variety of well known configurations, exemplary containment assembly configurations are described generally in U.S. Pat. No. 3,860,003 entitled “Contractible Side Portions for Disposable Diaper” which issued to Kenneth B. Buell on Jan. 14, 1975; and U.S. Pat. No. 5,151,092 entitled “Absorbent Article With Dynamic Elastic Waist Feature Having A Predisposed Resilient Flexural Hinge” which issued to Kenneth B. Buell et al., on Sep. 29, 1992; each of which is incorporated herein by reference.

The absorbent core 28 may be any absorbent member which is generally compressible, conformable, non-irritating to the wearer's skin, and capable of absorbing and retaining liquids such as urine and other certain body exudates. As shown in FIG. 1, the absorbent core 28 has an outer-facing (or garment-facing) side, a body-facing side, a pair of side edges, and a pair of waist edges. The absorbent core 28 may be manufactured in a wide variety of sizes and shapes (e.g., rectangular, hourglass, “T”-shaped, asymmetric, etc.) and from a wide variety of liquid-absorbent materials commonly used in disposable diapers and other absorbent articles such as comminuted wood pulp which is generally referred to as airfelt. Examples of other suitable absorbent materials include creped cellulose wadding; meltblown polymers including coform; chemically stiffened, modified or cross-linked cellulosic fibers; tissue including tissue wraps and tissue laminates; absorbent foams; absorbent sponges; superabsorbent polymers; absorbent gelling materials; or any equivalent material or combinations of materials.

The configuration and construction of the absorbent core 28 may vary (e.g., the absorbent core may have varying caliper zones, a hydrophilic gradient, a superabsorbent gradient, or lower average density and lower average basis weight acquisition zones; or may comprise one or more layers or structures). Further, the size and absorbent capacity of the absorbent core 28 may also be varied to accommodate wearers ranging from infants through adults. However, the total absorbent capacity of the absorbent core 28 should be compatible with the design loading and the intended use of the diaper 20.

One embodiment of the diaper 20 has an asymmetric, modified T-shaped absorbent core 28 having ears in the front waist region but a generally rectangular shape in the rear waist region. Exemplary absorbent structures for use as the absorbent core 28 of the present invention that have achieved wide acceptance and commercial success are described in U.S. Pat. No. 4,610,678 entitled “High-Density Absorbent Structures” issued to Weisman et al. on Sep. 9, 1986; U.S. Pat. No. 4,673,402 entitled “Absorbent Articles With Dual-Layered Cores” issued to Weisman et al. on Jun. 16, 1987; U.S. Pat. No. 4,888,231 entitled “Absorbent Core Having A Dusting Layer” issued to Angstadt on Dec. 19, 1989; and U.S. Pat. No. 4,834,735, entitled “High Density Absorbent Members Having Lower Density and Lower Basis Weight Acquisition Zones”, issued to Alemany et al. on May 30, 1989. The absorbent core may further comprise the dual core system containing an acquisition/distribution core of chemically stiffened fibers positioned over an absorbent storage core as detailed in U.S. Pat. No. 5,234,423, entitled “Absorbent Article With Elastic Waist Feature and Enhanced Absorbency” issued to Alemany et al., on Aug. 10, 1993; and in U.S. Pat. No. 5,147,345, entitled “High Efficiency Absorbent Articles For Incontinence Management” issued to Young, LaVon and Taylor on Sep. 15, 1992. All of these patents are incorporated herein by reference.

The topsheet 24 is preferably positioned adjacent the inner surface 62 of the absorbent core 28 and is preferably joined thereto and to the backsheet 26 by attachment means (not shown) such as those well known in the art. Suitable attachment means are described with respect to joining the backsheet 26 to the absorbent core 28. In a preferred embodiment of the present invention, the topsheet 24 and the backsheet 26 are joined directly to each other in the diaper periphery and are indirectly joined together by directly joining them to the absorbent core 28 by any suitable attachment means.

The topsheet 24 is preferably compliant, soft feeling, and non-irritating to the wearer's skin. Further, the topsheet 24 is preferably liquid pervious permitting liquids (e.g., urine) to readily penetrate through its thickness. A suitable topsheet 24 may be manufactured from a wide range of materials such as woven and nonwoven materials; polymeric materials such as apertured formed thermoplastic films, apertured plastic films, and hydroformed thermoplastic films; porous foams; reticulated foams; reticulated thermoplastic films; and thermoplastic scrims. Suitable woven and nonwoven materials can be comprised of natural fibers (e.g., wood or cotton fibers), synthetic fibers (e.g., polymeric fibers such as polyester, polypropylene, or polyethylene fibers) or from a combination of natural and synthetic fibers. The topsheet 24 is preferably made of a hydrophobic material to isolate the wearer's skin from liquids which have passed through the topsheet 24 and are contained in the absorbent core 28 (i.e. to prevent rewet). If the topsheet 24 is made of a hydrophobic material, at least the upper surface of the topsheet 24 is treated to be hydrophilic so that liquids will transfer through the topsheet more rapidly. This diminishes the likelihood that body exudates will flow off the topsheet 24 rather than being drawn through the topsheet 24 and being absorbed by the absorbent core 28. The topsheet 24 can be rendered hydrophilic by treating it with a surfactant. Suitable methods for treating the topsheet 24 with a surfactant include spraying the topsheet 24 material with the surfactant and immersing the material into the surfactant. A more detailed discussion of such a treatment and hydrophilicity is contained in U.S. Pat. No. 4,988,344 entitled “Absorbent Articles with Multiple Layer Absorbent Layers” issued to Reising, et al on Jan. 29, 1991 and U.S. Pat. No. 4,988,345 entitled “Absorbent Articles with Rapid Acquiring Absorbent Cores” issued to Reising on Jan. 29, 1991, each of which is incorporated by reference herein.

An alternative preferred topsheet comprises an apertured formed film. Apertured formed films are preferred for the topsheet because they are pervious to body exudates and yet non-absorbent and have a reduced tendency to allow liquids to pass back through and rewet the wearer's skin. Thus, the surface of the formed film which is in contact with the body remains dry, thereby reducing body soiling and creating a more comfortable feel for the wearer. Suitable formed films are described in U.S. Pat. No. 3,929,135, entitled “Absorptive Structures Having Tapered Capillaries”, which issued to Thompson on Dec. 30, 1975; U.S. Pat. No. 4,324,246 entitled “Disposable Absorbent Article Having A Stain Resistant Topsheet”, which issued to Mullane, et al. on Apr. 13, 1982; U.S. Pat. No. 4,342,314 entitled “Resilient Plastic Web Exhibiting Fiber-Like Properties”, which issued to Radel. et al. on Aug. 3, 1982; U.S. Pat. No. 4,463,045 entitled “Macroscopically Expanded Three-Dimensional Plastic Web Exhibiting Non-Glossy Visible Surface and Cloth-Like Tactile Impression”, which issued to Ahr et al. on Jul. 31, 1984; and U.S. Pat. No. 5,006,394 “Multilayer Polymeric Film” issued to Baird on Apr. 9, 1991. Each of these patents are incorporated herein by reference.

The backsheet 26 of the present invention comprises a nonwoven web 90 positioned at the outermost portion of the absorbent article, which covers at least a portion of the outermost portion of the absorbent core of the article. In preferred embodiments, the nonwoven web 90 is present on at least 30%, more preferably at least 70%, most preferably at least 90% of the area of the outer-facing surface of the absorbent article. In preferred embodiments, the backsheet 26 further comprises a plastic film 27 having an outer-facing surface and a body-facing surface, and the nonwoven web 90 is joined with the outer-facing surface of the plastic film to form a laminate. The nonwoven web may be joined to the plastic film by any suitable attachment means known in the art. For example, the nonwoven web may be secured to the plastic film by a uniform continuous layer of adhesive, a patterned layer of adhesive, or an array of separate lines, spirals, or spots of adhesive. Suitable adhesives include a hotmelt adhesive obtainable from Nitta Findley Co., Ltd., Osaka, Japan as H-2476-01, and a hotmelt adhesive obtainable from H.B. Fuller Japan Co., Ltd., Osaka, Japan as JM-6064. Preferably, the density of the adhesive applied between the nonwoven web and the plastic film is from about 0.05 g/m² to about 7.0 g/m², more preferably from about 0.1 g/m² to about 5.0 g/m², most preferably from about 0.2 g/m² to about 1.5 g/m².

The plastic film is preferably impervious to liquids (e.g., urine) and is preferably manufactured from a thin plastic film. However, the plastic film permits vapors to escape from the diaper 20. In a preferred embodiment, a microporous polyethylene film is used for the plastic film. A suitable microporous polyethylene film is manufactured by Mitsui Toatsu Chemicals, Inc., Nagoya, Japan and marketed in the trade as Espoir No.

A suitable material for the plastic film is a thermoplastic film having a thickness of from about 0.012 mm (0.5 mil) to about 0.051 mm (2.0 mils), preferably comprising polyethylene or polypropylene. Preferably, the plastic film has a basis weight of from about 5 g/m² to about 35 g/m². However, it should be noted that other flexible liquid impervious materials may be used. As used herein, the term “flexible” refers to materials which are compliant and which will readily conform to the general shape and contours of the wearer's body. In preferred embodiments, the backsheet 26 of the present invention may comprise a single member such as the film described above, or may comprise a number of materials joined together to form the plastic film 27.

Preferably, the nonwoven web 90 may cover all or substantially all of the outer-facing surface 70 of the plastic film 27, or may cover only discrete predetermined portions. In a preferred embodiment, the nonwoven web 90 covers all or substantially all of the plastic film 27 in order to provide the diaper with a cloth-like appearance and feel. Further, the nonwoven web 90 may provide the diaper with a low cost landing zone capable of engaging the hooks of a hook and loop type fastener. (Such a landing zone could be utilized as a portion of a primary fastening system or as a means for disposing of a soiled diaper.)

In a preferred embodiment, the plastic film 27 exists only in the containment assembly area 22 (and does not exist the side panel areas 30), while the nonwoven web 90 exists the both of the containment assembly area 22 and the side panel areas 30. The nonwoven web 90 covers all of the outer-facing surface 70 of the plastic film 27.

The nonwoven web 90 is preferably air pervious. The nonwoven web may comprise natural fibers (e.g. cotton or wood fibers), or may comprise fibers of polyethylene, polypropylene, polyester, polyethylene terephthalate, or any combination of such fibers. Further, the nonwoven web may be carded, spunmelt, meltblown or air-through bonded or have any other characteristic or be manufactured in any manner known in the art. Preferably, the nonwoven web is comprised of sufficient thermoplastic material to allow for thermal bonding of the material to other components of the diaper.

An especially preferred nonwoven web is a spunbonded nonwoven web, preferably made of bi-component fibers. Preferably, the bi-component fiber contains a polyethylene and a polypropylene. More preferably, the bi-component fiber has a core of the polypropylene and a sheath of the polyethylene. In preferred embodiments, the bi-component fiber has from about 55% to about 95% by weight of the polyethylene. Most preferably, the bi-component fiber has from about 70% to about 90% by weight of the polyethylene.

In preferred embodiments, the spunbonded nonwoven web is placed in the disposable absorbent article so that the fiber direction of the spunbonded bi-component plastic fibers is aligned with the longitudinal direction of the disposable absorbent article. Preferably, the spunbonded nonwoven web has a tensile strength of at least 80 gf/cm, more preferably of at least 180 gf/cm in the traverse direction of the disposable absorbent article.

An alternative preferred nonwoven web is a carded nonwoven web, preferably made of bi-component fibers. Preferably, the bi-component fiber contains a polyethylene and a polyethylene terephthalate. Preferably, the bi-component fiber has a core of the polyethylene terephthalate and a sheath of the polyethylene. In preferred embodiments, the bi-component fiber has from about 50% to about 95% by weight of the polyethylene. Most preferably, the bi-component fiber has from about 55% to about 95% by weight of the polyethylene.

In a further alternative embodiment, the bi-component fiber may contain different types of polypropylene. More preferably, the bi-component fiber has a core of the polypropylene which has a higher melting point and a sheath of the polyethylene which has a lower melting point.

In a preferred embodiment, the nonwoven web is a carded nonwoven web obtainable from Havix Co., LTD., Gifu, Japan as E-2341. The nonwoven web is made of bi-component fibers of a polyethylene (PE) and a polyethylene terephthalate (PET). The ratio of PE/PET is about 60/40. The PE/PET bi-component fiber has the dimension of 2d×51 mm.

In an alternative preferred embodiment, the nonwoven web is a spunbonded nonwoven web obtainable from Mitsui Petrochemical Industries, Ltd., Tokyo, Japan. The nonwoven web is made of bi-component fibers of a polyethylene (PE) and a polypropylene (PP). The ratio of PE/PP is about 80/20. The PE/PP bi-component fiber has the thickness is approximately 2.3d.

The backsheet 26 is preferably positioned adjacent the outer surface of the absorbent core 28 and is preferably joined thereto by any suitable attachment means known in the art. For example, the backsheet 26 may be secured to the absorbent core 28 by a uniform continuous layer of adhesive, a patterned layer of adhesive, or an array of separate lines, spirals, or spots of adhesive. Adhesives which have been found to be satisfactory are manufactured by H. B. Fuller Company of St. Paul, Minn. and marketed as HL-1258. An example of a suitable attachment means comprising an open pattern network of filaments of adhesive is disclosed in U.S. Pat. No. 4,573,986 entitled “Disposable Waste-Containment Garment”, which issued to Minetola et al. on Mar. 4, 1986. Another suitable attachment means comprising several lines of adhesive filaments swirled into a spiral pattern is illustrated by the apparatus and methods shown in U.S. Pat. No. 3,911,173 issued to Sprague, Jr. on Oct. 7, 1975; U.S. Pat. No. 4,785,996 issued to Ziecker, et al. on Nov. 22, 1978; and U.S. Pat. No. 4,842,666 issued to Werenicz on Jun. 27, 1989. Each of these patents are incorporated herein by reference. Alternatively, the attachment means may comprise heat bonds, pressure bonds, ultrasonic bonds, dynamic mechanical bonds, or any other suitable attachment means or combinations of these attachment means as are known in the art.

Embodiments of the present invention are also contemplated wherein the absorbent core is not joined to the backsheet 26, and/or the topsheet 24 in order to provide greater extensibility in the front waist region 46 and the rear waist region 44. Alternative embodiments are contemplated wherein an additional member, such as a liquid impervious barrier material(s) (not shown), is positioned between the outer surface 64 of the absorbent core 28 and the backsheet 28. Any such barrier member may or may not be joined to the absorbent core 28. Further, the backsheet 26 may or may not be joined to any barrier material(s) that are positioned between the backsheet 26 and the absorbent core 28.

In one aspect of the present invention, the backsheet has a hand value of Koshi (Stiffness) of less than about 11.0, a hand value of Shari (Crispness) of from about 5.0 to about 7.0, and a hand value of Fukurami (Fullness and Softness) less than about 0.5. Preferably, the backsheet has a hand value of Koshi of less than about 7.0. In preferred embodiments, the backsheet has a hand value of Shari is from about 5.5 to about 6.5. Preferably, the backsheet has a hand value of Fukurami of less than about 0.1.

The hand value of Koshi approximates the feeling attributable to the bending stiffness of the backsheet. The springy property of the backsheet material enhances this feeling. A backsheet having a compact weaving density, which is woven with springy and elastic yarns enhances this feeling. The hand value of Shari approximates the feeling, which results from the crisp and rough surface of the backsheet. This feeling is caused by hard and strongly twisted yarns. The hand value of Fukurami approximates the feeling, which results from the bulky, rich and well formed backsheet.

Thus, the backsheet of the present invention has suitable ranges of hand values, which enhances surface smoothness and softness. For example, the backsheet of the invention reduces frictions between the backsheet and the wearer's skin in order to prevent skin rash at the wearer's legs. Further, the backsheet of the invention reduces frictions caused between the backsheet and the wearer's clothes. Thus, the generation of noise can be prevented to improve the wearer's comfort.

In a preferred embodiment, the backsheet has a fuzz level of less than about 0.24 mg/cm², more preferably of less than about 0.14 mg/cm², and most preferably of less than about 0.05 mg/cm². The fuzz level approximates the amount of untangled fibers, which protrude from the surface of the backsheet. Higher fuzz level results in greater skin irritation as well as a greater itching sensation to the skin. The fuzz level relates to the quantity of untangled fibers which protrude from the surface of the backsheet. The fuzz level also corresponds to the propensity of untangled fibers to get removed from the surface of the backsheet.

In more preferred embodiments, the backsheet has a mean value of coefficient of friction (MIU) of less than about 0.21, more preferably less than about 0.18. Lower values of MIU result in lower frictions between the backsheet and the wearer's skin, and between the backsheet and the wearer's clothes.

An especially preferred nonwoven web is a spunbonded nonwoven web, preferably made of bi-component fibers. In preferred embodiments, the spunbonded nonwoven web has a hand value of Koshi of less than about 16.0, a hand value of Shari of from about 0.5 to about 9.5, and a hand value of Fukurami of less than about 5.0. In a preferred embodiment, the spunbonded nonwoven web has a fuzz level of less than about 1.0.

These hand values and levels are calculated based on the physical properties which are obtained from the following measurements. The physical properties include 1) Tensile property; 2) Bending property; 3) Surface property; 4) Shearing property; 5) Compression property; and 6) Weight and thickness. These properties include in total sixteen (16) characteristic values or detailed properties as indicated in the Table I.

TABLE I

| Property | Symbols | Property | Unit | Remarks |
|---|---|---|---|---|
| Tensile | LT | linearity of load- | none | LT = 1: completely linear |
| | | extension curve | | and |
| | | | | LT = 0: extremely non-linear |
| | WT | tensile energy per | gf · | Higher value of WT corresponds to |
| | | unit area | cm/cm² | higher extensibility. |
| | RT | tensile resilience | % | RT = 100%: completely elastic |
| | | | | RT = 0%: completely inelastic |
| | EM | extensibility | % | Strain at maximum load |
| | | | | (= 50 gf/cm) |
| Bending | B | bending rigidity | gf · | Bending rigidity per unit width of |
| | | | cm²/cm | fabric. |
| | 2HB | hysteresis of bending | gf · | Hysteresis of bending moment |
| | | moment | cm/cm | observed in the bending moment- |
| | | | | curvature relationship. A larger |
| | | | | value of 2HB means a greater |
| | | | | fabric inelasticity |
| Shearing | G | shear stiffness | gf/cm | |
| | | | degree | |
| | 2HG | hysteresis of shear | gf/cm | |
| | | force at 0 degree of | | |
| | | shear angle | | |
| Compression | LC | linearity of | none | LC = 1: completely linear |
| | | compression- | | LC = 0: completely non-linear |
| | | thickness curve | | |
| | WC | compressional energy | gf · | A larger value of WC corresponds |
| | | | cm/cm² | to higher compressibility. |
| | RC | compressional | % | RC = 100%: elastic |
| | | resilience | | and |
| | | | | RC = 0%: completely inelastic |
| Surface | MIU | coefficient of friction | none | Higher value corresponds to higher |
| | | | | friction. |
| | MMD | mean deviation of | none | Higher value corresponds to larger |
| | | MIU | | variation of friction. |
| | SMD | geometrical | μm | Higher value corresponds to |
| | | roughness | | geometrically rough surface. |
| Weight | W | sample weight | mg/cm² | |
| and | Tₒ | sample thickness | mm | Thickness at pressure of 0.5 gf/cm² |
| Thickness | | | | |

The sixteen characteristic values are obtained by the measurement and analytical methods described in the next section. Similar (although not identical) measurement and analytical methods for fabric are known and described, for example, in the Chapter IV of the text book, by Sueo Kawabata, entitled “The Standardization and Analysis of Hand Evaluation (2nd. Edition)”, published by the Textile Machinery Society of Japan, July 1980. The disclosure of this book is incorporated herein by reference. Based on the sixteen characteristic values obtained from the measurements, the hand values of Koshi, Shari and Fukurami are obtained according to the following analytical methods. The fuzz level is measured by the specific method described in the later section.

### 2. Measurement and Calculation Methods for Hand Values and Fuzz Level

### 1) Tensile Property:

The backsheet sample is subjected to applied unidirectional extension stress up to a maximum load of 50 gf/cm and then allowed to return to its initial state. The speed of the deformation is 0.1 mm/s. The effective dimension of the sample is 20 cm in width and 2.5 cm in length (rectangular). As a result, the tensile property curve as shown in FIG. 4 is obtained by the measurement. The horizontal axis shows the strain (%) and the vertical axis shows the stress (gf/cm). The characteristic values of LT, WT, and RT are calculated as follows:

LT=(Sa+Sb)/(Sa+Sb+Sc) (1)

WT=Sa+Sb (2)

RT=Sb/(Sa+Sb) (3)

where Sa, Sb, Sc are defined by the areas shown in FIG. 4.

### 2) Bending Property:

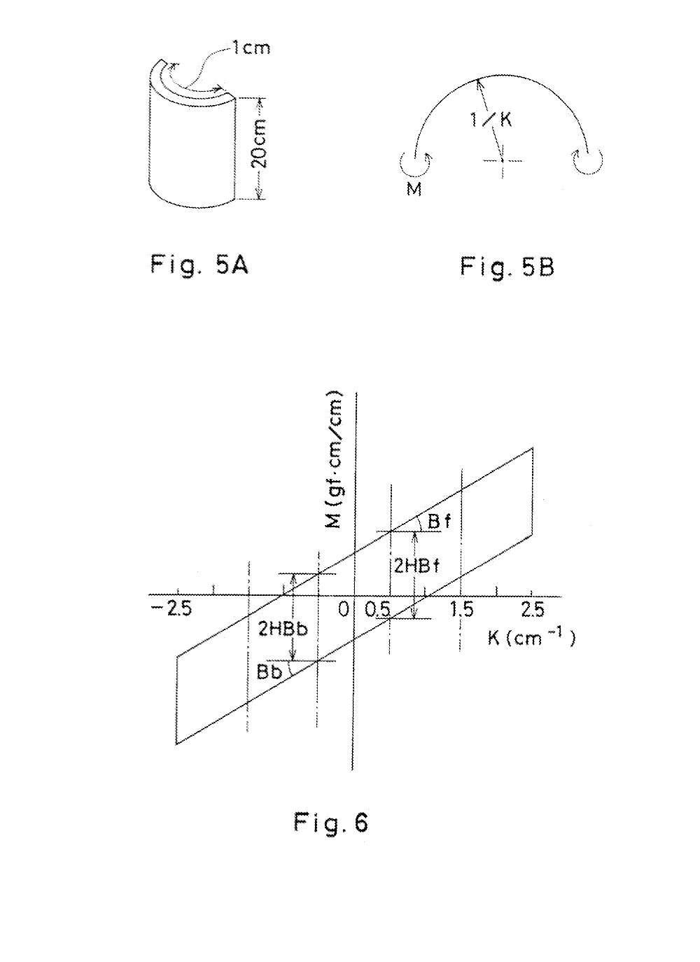

The deformation mode is obtained by bending the sample between a curvature K=−2.5 cm⁻¹ and K=2.5 cm⁻¹. The deformation mode is a measure of the force required to bend the sample. The effective dimension of the sample for this measurement is 2.0 cm in length and 1.0 cm in width (rectangular). The sample is bent as shown in FIGS. 5A and 5B. The bending rate is 0.5 cm⁻¹/sec. As a result, a bending histeresis curve as shown in FIG. 6 is obtained. The horizontal axis shows the curvatures K cm⁻¹ and the vertical axis shows the moment M (gf·cm/cm). The values of B and 2HB are calculated as follows:

B=(Bf+Bb)/2 (4)

where Bf and Bb are the slopes of the histeresis curves between K=0.5 cm⁻¹ and 1.5 cm⁻¹ and K=−0.5 cm⁻¹ and −1.5 cm⁻¹ respectively.

2HB=(2HBf+2HBb)/2 (5)

where 2HBf and 2HBb are the histeresis differences at K=0.5 cm⁻¹ and −0.5 cm⁻¹, respectively.

### 3) Surface Property:

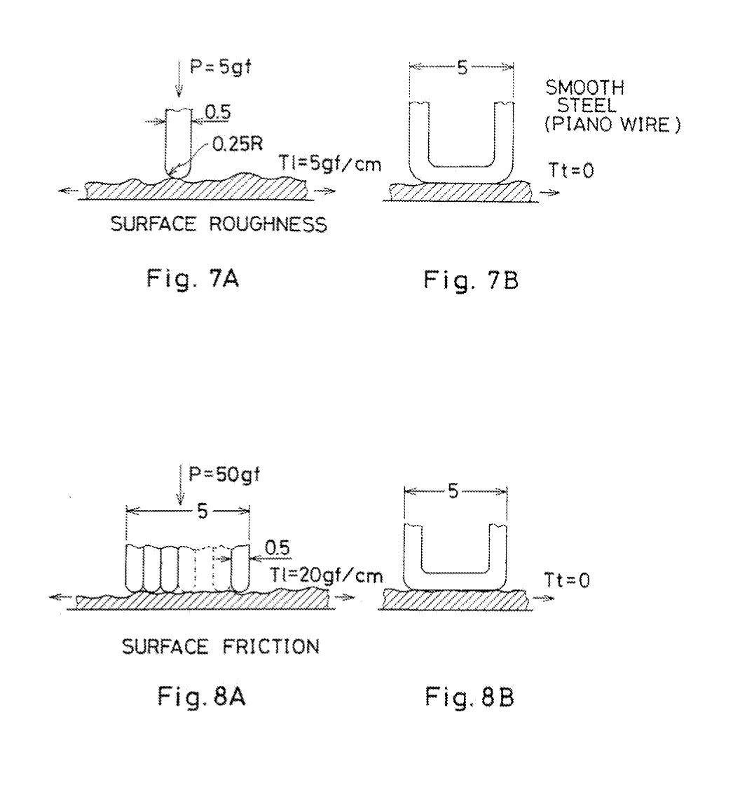

To measure the surface roughness of the sample, a pianowire is prepared and bent as shown in FIGS. 7A and 7B. 5.0 gf (allowance, ±0.5 gf) of the contact force is applied by a spring having a spring constant of 25±1 gf/mm. The natural frequency of the system should be more than 30 Hz when the contactor does not contact the sample.

The friction between the surfaces of the sample and a contactor is measured under a constant contact pressure. Surface friction should be measured by using the contactor shown in FIGS. 8A and 8B. The surface of the contactor is covered by ten parallel and stacked piano steel wires. The ten pieces of the same wires are placed on the surface of specimen. A compression force of 50 gf by dead weight is applied to the surface of the sample through the contactor.

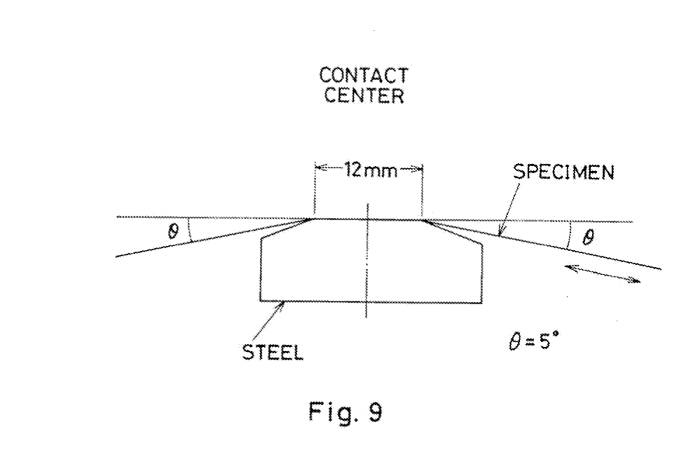

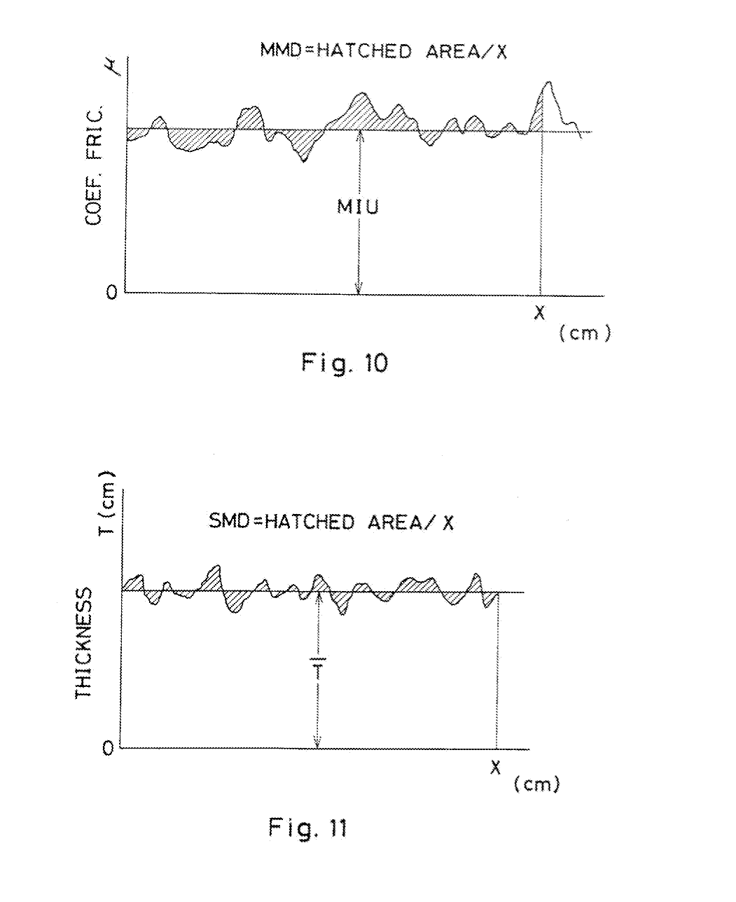

In the both of the roughness and friction measurements, the specimen displaced a distance of 2 cm at a constant velocity of 0.1 cm/sec on a smooth steel plate placed horizontally where the tension of the specimen is kept at 5.0 gf/cm (force per unit length) and the contactor is kept its position. The dimension of the plate is shown in FIG. 9. The variations of the surface coefficient of friction μ and the sample thickness T that are obtained are shown in FIGS. 10 and 11.

Consequently, the values of MIU, MMD and SMD are obtained from the following expressions:

$$\begin{matrix}MIU = \frac{1}{X} {\int }_{0}^{x} μ d x , & ( 6 ) \\ MIU = \frac{1}{X} {\int }_{0}^{x} \vert μ - {μ}^{′} \vert d x , & ( 7 ) \\ SMD = \frac{1}{X} {\int }_{0}^{x} \vert T - {T}^{′} \vert d x , & ( 8 )\end{matrix}$$

where μ; frictional force/compressional force

μ′; mean value of μ

x; displacement of the contactor on the surface of sample

X; 2 cm is taken in this measurement

T; Thickness of the sample at position x

T′; Mean value of T

### 4) Shearing Property:

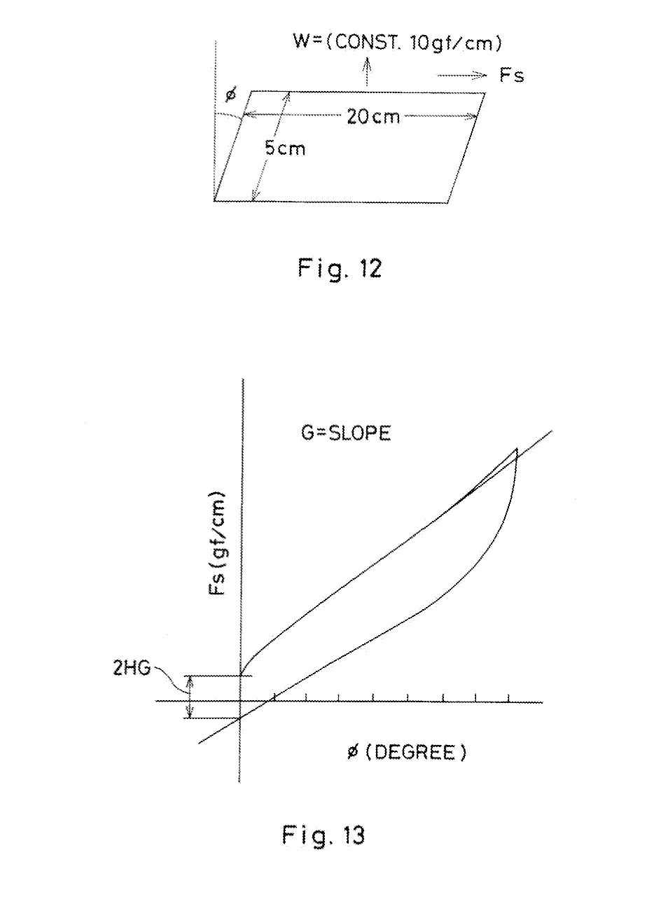

A constant extension force of 5 gf/cm is applied on a longitudinal direction of the sample and then a shear force Fs is applied to the sample plane along the transverse direction of the sample up to the shear angle ø=4° as shown in FIG. 12. Then, the sample shear deformation is recovered by reducing the shear angle back to zero. The effective dimension of the sample is 20 cm in width and 5 cm in length. The relationship between Fs and ø that is obtained is shown in FIG. 13. The value of 2HG is obtained from the hysteresis curve at ø=0°. The value of G is calculated as follows:

G=(Gf+Gb)/2 (9)

where Gf and Gb are the average slopes of the curve between ø=0.5° and 5° and between ø=−0.5° and −5° respectively.

### 5) Compression Property:

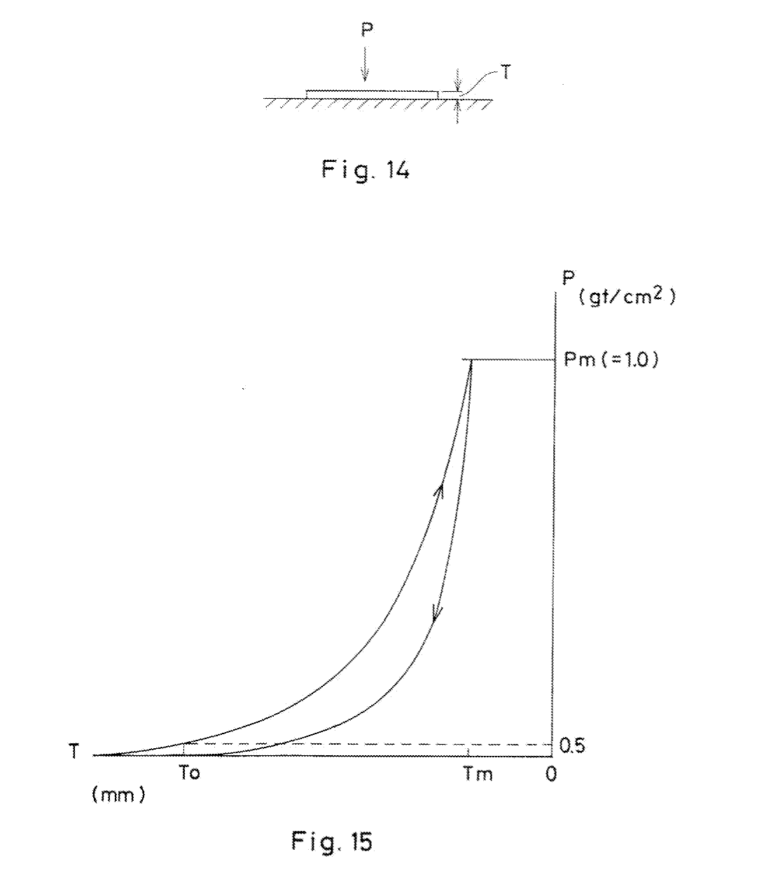

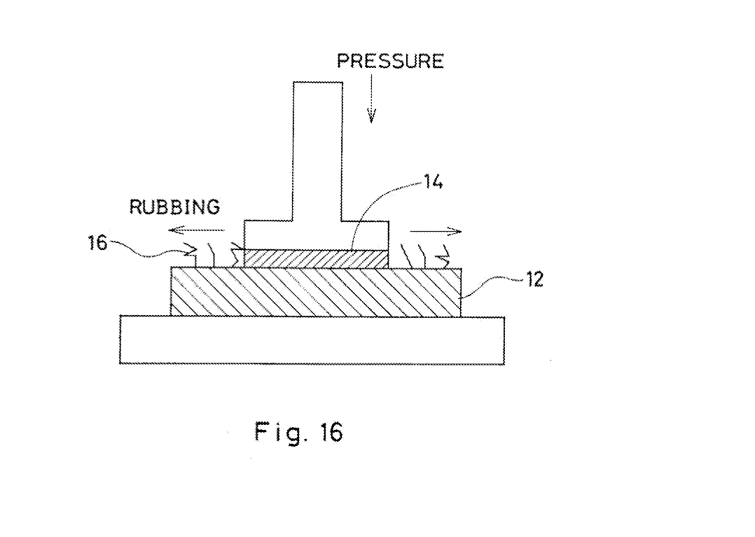

For this test, the effective dimension of the sample is 2.5 cm long and 2.0 cm in width. The longitudinal direction of the sample is taken along either warp or weft direction. 2 cm² of a circled area of the sample is compressed by two circular-plates of steel having 2 cm² area (FIG. 14). The velocity of the compression is 20 micron/sec and when the pressure reaches 10 g/cm², the recovery process is measured at the same velocity. The values of LC, WC and RC are obtained by the following expressions:

$$\begin{matrix}LC = WC / WOC & ( 10 ) \\ WC = {\int }_{Tm}^{To} P d T & ( 11 ) \\ RC = {WC}^{′} / WC & ( 12 )\end{matrix}$$

where

T; Thickness of the sample (cm).

To; Thickness of the sample at a pressure of 0.5 gf/cm².

Tm; Thickness of the sample at maximum pressure Pm which is:
Pm=10 gf/cm²

WOC;=Pm(To−Tm)/2 (13)

WC′; Recovering energy given by the pressure of the recovering process, P′ such as

$${WC}^{′} = {\int }_{Tm}^{To} {P}^{′} d T \qquad ( 14 )$$

### 6) Weight and Thickness:

The value of T mm is measured as the thickness when the compressional property is measured (P=0.5 gf/cm²). The value of W g/m² is measured as the weight per unit area of the sample.

### (2) Calculation of Hand Values

The hand values of Koshi, Shari and Fukurami are obtained from the following expression (15) by applying the sixteen characteristic values obtained from the measurements. The calculation using the expression (15) is conducted according to the Knit High Sensivity Condition (KN-403-KTV).

$$HV = Co + {\sum }_{16}^{i = 1} \{ Ci \cdot ( Xi - {Xi}^{′} ) / σ i \} \qquad ( 15 )$$

where HV is the hand value.

The numbers and constants to be applied to the equation are indicated in the Tables II and III.

TABLE II

| Property | i | Xi | Xi′ | i |
|---|---|---|---|---|
| | 0 | | | |
| Tensile | 1 | LT | 0.7756 | 0.0679 |
| | 2 | log WT | 0.6808 | 0.2557 |
| | 3 | log RT | 1.5952 | 0.0639 |
| Bending | 4 | log B | −1.6441 | 0.3288 |
| | 5 | log 2HB | −1.5180 | 0.3213 |
| Shearing | 6 | log 2G | −0.4000 | 0.1276 |
| | 7 | log 2HG | 0.0444 | 0.1486 |
| | 8 | log 2HG5 | 0.0444 | 0.1486 |
| Compression | 9 | LC | 0.6337 | 0.0692 |
| | 10 | log WC | −0.9937 | 0.1526 |
| | 11 | RC | 38.1224 | 5.6815 |
| Surface | 12 | log HIU | −0.5952 | 0.0861 |
| | 13 | log HMD | −1.5999 | 0.2018 |
| | 14 | log SMD | 0.9280 | 0.1999 |
| Weight and | 15 | log T | 0.0638 | 0.1361 |
| Thickness | 16 | W | 17.3383 | 5.0040 |

TABLE III

| | | |
|---|---|---|
| | For | |
| For Koshi | Fukurami | For Shari |

| i | Ci | i | Ci | i | Ci |
|---|---|---|---|---|---|
| 0 | 4.4473 | 0 | 4.5531 | 0 | 4.8480 |
| 6 | 0.9934 | 9 | −0.1760 | 14 | 1.1399 |
| 7 | −0.0264 | 10 | 1.9067 | 13 | 0.1485 |
| 8 | 0.4165 | 11 | 0.7942 | 12 | −0.1527 |
| 4 | 0.5064 | 15 | −0.0193 | 10 | −0.5692 |
| 5 | 0.3654 | 16 | 0.4399 | 11 | −0.2623 |
| 15 | −0.1568 | 12 | −0.1182 | 9 | 0.1401 |
| 16 | 0.2789 | 13 | −0.4141 | 4 | 0.5975 |
| 1 | −0.2437 | 14 | 0.1194 | 5 | −0.1113 |
| 2 | −0.1740 | 1 | −0.0169 | 1 | 0.1786 |
| 3 | 0.0931 | 2 | 0.2347 | 3 | 0.0492 |
| 9 | −0.1255 | 3 | −0.1000 | 2 | −0.0307 |
| 10 | 0.1252 | 6 | −0.3254 | 16 | −0.1966 |
| 11 | 0.0119 | 7 | −0.4482 | 15 | −0.0113 |
| 12 | −0.0125 | 8 | 0.8427 | 8 | 0.1770 |
| 13 | 0.1037 | 4 | −0.2441 | 6 | −0.0284 |
| 14 | 0.0276 | 5 | 0.1412 | 7 | 0.0135 |

### (3) Measurement and Analysis Equipment

An example of preferred measurement and analysis equipment is the type KES FB1-FB4 which is available form Kato Tech Co., LTD., Kyoto Japan. The backsheet sample to be used by this equipment is a square sheet of 20 cm×20 cm. The measurement and analysis are conducted on at least three (3) samples, more preferably at least ten (10) samples.

### (4) Fuzz Level Measurement

To measure the quantity of untangled fibers that protrude from the surface of the sample, the face of the sample 12 is rubbed against the face of sandpaper 14 for 29 seconds at 0.7 Hz to cut or loosen the untangled fibers 16. 18.1 gf/cm² of pressure is applied to the sample 12. An example of the equipment is shown in FIG. 16. The cut fibers produced by this action are collected by a removal tape and quantified with an analytical balance. The fuzz level is defined as the weight of the fibers collected per unit area (mg/cm²).

An example of equipment available is Sutherland Ink Rub Tester. 18.1 gf/cm² of pressure is applied to the sample. This apparatus abrades a 6.5 cm×15 cm piece of sample with a 15 cm×5.1 cm piece of sandpaper (Matelite K224 Cloth Sandpaper Grit 320-J, Norton Co., Troy, N.Y.). The rub cycle is 20 times at 0.7 cycle/sec. The fibers (fuzz) are removed using two 15 cm×5.1 cm pieces of removal tape (3M Scotch Carton Sealing Tape, J A Kindel, Cincinnati, Ohio) from both the sandpaper and sample.

### 3. Examples

### Example I

23 g/m² of a carded nonwoven web obtainable from Havix Co., LTD., Gifu, Japan as E-2341 is prepared. The nonwoven web is made of bi-component fibers of a polyethylene (PE) and a polyethylene terephthalate (PET). The nonwoven web is joined to 25 g/m² of a microporous polyethylene (PE) film obtainable from Mitsui Toatsu Chemicals, Inc., Nagoya, Japan as Espoir No. by using 5 g/m² of a hotmelt adhesive obtainable from Nitta Findley Co., Ltd., Osaka, Japan as H-2476-01 with a meltblown pattern (Nordson K.K., Tokyo, Japan). The PE/PET bi-component fiber has the dimension of 2d×51 mm. The ratio of PE/PET is about 60/40. The open time and temperature of hotmelt adhesive before the application is 0.5 sec and 160° C., respectively.

This backsheet sample has hand values of Koshi, Shari, and Fukurami are 10.6, 0.2, and 6.3, respectively. The fuzz level is 0.31 mg/cm². The mean value of coefficient of friction (MIU) is 0.19.

### Example II

23 g/m² of a spunbonded nonwoven web obtainable from Mitsui Petrochemical Industries, Ltd., Tokyo, Japan is prepared. The nonwoven web is made of bi-component fibers of a polyethylene (PE) and a polypropylene (PP). The nonwoven web is joined to 25 g/m² of a microporous polyethylene (PE) film obtainable from Mitsui Toatsu Chemicals, Inc., Nagoya, Japan as Espoir No. by using 1.5 g/m² of a hotmelt adhesive obtainable from H.B. Fuller Japan Co., Ltd., Osaka, Japan as JM-6064 with a random dot pattern (Porous Coating, Nordson K.K., Tokyo, Japan). The PE/PP bi-component fiber has the thickness of approximately 2d. The ratio of PE/PP is about 80/20. The open time and temperature of hotmelt adhesive before the application is about 0.5 sec and about 160° C., respectively. The mean value of coefficient of friction (MIU) is 0.21.

This backsheet sample has hand values of Koshi, Shari, and Fukurami are 9.6, −2.4, and 6.6, respectively. The fuzz level is 0.24 mg/cm. The spunbonded nonwoven web has a tensile strength of 185 gf/cm at 12.7 cm/min of sample displacement speed (Model 4301: Instron Japan Co., Ltd., Kanagawa, Japan).

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable absorbent article having a first waist region, a second waist region and a crotch region connecting the first and second waist regions, a longitudinal centerline and a transverse centerline, said disposable absorbent article comprising:
a containment assembly comprising a liquid pervious topsheet, a liquid impervious backsheet, an absorbent core disposed between said liquid pervious topsheet and said liquid impervious backsheet, and a pair of elasticized leg cuffs that are disposed inboard of said side edges of said containment assembly, said liquid impervious backsheet comprising a plastic film having inner and outer surfaces;
a first side panel joined to said containment assembly at said first waist region, wherein said first side panel is disposed on a left portion of the absorbent article relative to said longitudinal centerline;
a second side panel joined to said containment assembly at said first waist region, wherein said second side panel is disposed on a right portion of the absorbent article relative to said longitudinal centerline;
a first securement member that extends outboard of said first side panel, said first securement member comprising a plurality of hook;
a second securement member that extends outboard of said second side panel, said second securement member comprising a plurality of hook; and
a landing zone comprising a nonwoven web, wherein said landing zone is disposed in said second waist region and covers only a discrete predetermined portion of the outer-facing surface of said plastic film and wherein said nonwoven web comprises bi-component fibers.

2. The disposable absorbent article of claim 1 wherein said bi-component fibers comprise a core and a sheath.

3. The disposable absorbent article of claim 2 wherein said core of said bi-component fibers comprises a polypropylene and said sheath of said bi-component fibers comprises a polyethylene.

4. The disposable absorbent article of claim 3 wherein said bi-component fibers comprise spunbond fibers.

5. The disposable absorbent article of claim 1 wherein said nonwoven web has a tensile strength of at least 80 gf/cm in a transverse direction of said disposable absorbent article.

6. The disposable absorbent article of claim 1 wherein the bi-component fibers have from about 55% to about 95% by weight of polyethylene.

7. The disposable absorbent article of claim 1 wherein said first and second side panels are elastically extensible.

8. The disposable absorbent article of claim 7 wherein said first and second side panels comprise a zero strain stretch laminate.

9. The disposable absorbent article of claim 1 wherein said plastic film comprises polyethylene.

10. The disposable absorbent article of claim 1 wherein said landing zone overlaps with said absorbent core.

11. The disposable absorbent article of claim 1 wherein said landing zone and said absorbent core have a transverse length and wherein the transverse length of said landing zone is greater than the transverse length of said absorbent core.

12. The disposable absorbent article of claim 1 wherein said landing zone overlaps with each of said elasticized leg cuffs.

13. The disposable absorbent article of claim 1 wherein said landing zone so that the fiber direction of the bi-component fibers is substantially aligned with the longitudinal centerline of the disposable absorbent article.

14. A disposable absorbent article having a first waist region, a second waist region and a crotch region connecting the first and second waist regions, a longitudinal centerline and a transverse centerline, said disposable absorbent article comprising:
a containment assembly comprising a liquid pervious topsheet, a liquid impervious backsheet, an absorbent core disposed between said liquid pervious topsheet and said liquid impervious backsheet, and a pair of elasticized leg cuffs that are disposed inboard of said side edges of said containment assembly, said liquid impervious backsheet comprising a plastic film having inner and outer surfaces;
a first side panel joined to said containment assembly at said first waist region, wherein said first side panel is disposed on a left portion of the absorbent article relative to said longitudinal centerline;
a second side panel joined to said containment assembly at said first waist region, wherein said second side panel is disposed on a right portion of the absorbent article relative to said longitudinal centerline;
a third said panel joined to said containment assembly at said second waist region, wherein said third side panel is disposed on a left portion of the absorbent article relative to said longitudinal centerline;
a fourth said panel joined to said containment assembly at said second waist region, wherein said fourth side panel is disposed on a right portion of the absorbent article relative to said longitudinal centerline;
a first securement member that extends outboard of said first side panel, said first securement member comprising a plurality of hook;
a second securement member that extends outboard of said second side panel, said second securement member comprising a plurality of hook; and
a landing zone comprising a nonwoven web, wherein said landing zone is disposed in said second waist region and covers only a discrete predetermined portion of the outer-facing surface of said plastic film and wherein said nonwoven web comprises bi-component fibers.

15. The disposable absorbent article of claim 14 wherein said bi-component fibers comprise a core and a sheath.

16. The disposable absorbent article of claim 15 wherein said core of said bi-component fibers comprises a polypropylene and said sheath of said bi-component fibers comprises a polyethylene.

17. The disposable absorbent article of claim 16 wherein said bi-component fibers comprise spunbond fibers.

18. The disposable absorbent article of claim 14 wherein said nonwoven web has a tensile strength of at least 80 gf/cm in a transverse direction of said disposable absorbent article.

19. The disposable absorbent article of claim 14 wherein the bi-component fibers have from about 55% to about 95% by weight of polyethylene.

20. The disposable absorbent article of claim 14 wherein said first and second side panels are elastically extensible.

21. The disposable absorbent article of claim 20 wherein said first and second side panels comprise a zero strain stretch laminate.

22. The disposable absorbent article of claim 14 wherein said plastic film comprises polyethylene.

23. The disposable absorbent article of claim 14 wherein said landing zone overlaps with said absorbent core.

24. The disposable absorbent article of claim 14 wherein said landing zone and said absorbent core have a transverse length and wherein the transverse length of said landing zone is greater than the transverse length of said absorbent core.

25. The disposable absorbent article of claim 14 wherein said landing zone overlaps with each of said elasticized leg cuffs.

26. The disposable absorbent article of claim 14 wherein said landing zone so that the fiber direction of the bi-component fibers is substantially aligned with the longitudinal centerline of the disposable absorbent article.
